(19) United States
(12) Patent Application Publication
(10) Pub. No.: US 2016/0243201 A1
(43) Pub. Date: Aug. 25, 2016

(54) METHODS AND COMPOSITIONS FOR INCREASING HEPCIDIN EXPESSION USING MODIFIED IRON BINDING/RELEASING TRANSFERRIN

(71) Applicant: NEW YORK BLOOD CENTER, INC., New York, NY (US)

(72) Inventor: Yelena Z. Ginzburg, New York, NY (US)

(21) Appl. No.: 15/033,290

(22) PCT Filed: Nov. 4, 2014
(86) PCT No.: PCT/US14/63928

§ 371 Date: Apr. 29, 2016

Related U.S. Application Data
(60) Provisional application No. 61/900,222, filed on Nov. 5, 2013.

(51) Int. Cl. A61K 38/40; A61K 9/00

(52) U.S. Cl. CPC A61K 38/40; A61K 9/0019

(57) ABSTRACT

Provided are methods for increasing hepcidin expression, treating a disorder associated with iron overload, decreasing non-transferrin bound iron (NTBI), reducing spleen size, ameliorating ineffective erythropoiesis, decreasing iron uptake by erythroid cells, and increasing transferrin receptor 1 (TfR1) expression in a subject in need thereof, comprising administering to said subject a therapeutically effective amount of a modified iron binding/releasing transferrin, wherein said modified iron binding/releasing transferrin comprises an N-lobe and a C-lobe, and wherein one of said lobes binds iron, and wherein one of said lobes has a decreased binding affinity for iron.

### CROSS REFERENCE TO RELATED APPLICATIONS

The present application claims the benefit under 35 USC §119(e) to U.S. Provisional Patent Application 61/900,222 filed Nov. 5, 2013, the entire contents of which is incorporated by reference herein.

### STATEMENT REGARDING FEDERALLY SPONSORED RESEARCH OR DEVELOPMENT

This invention was made with United States Government support of Grant No. 5K08HL105682-03 awarded by the National Institutes of Health. The United States Government has certain rights in this invention.

### FIELD OF THE INVENTION

The invention relates to methods and compositions for increasing hepcidin expression using modified iron binding/releasing transferrin, as well as to method and compositions for treating diseases and disorders associated with insufficient hepcidin expression and/or ineffective erythropoiesis.

### BACKGROUND

β-thalassemias are caused by mutations in the β-globin gene resulting in reduced or absent β-chain synthesis. A relative excess of α-globin chain synthesis leads to increased erythroid precursor apoptosis causing ineffective erythropoiesis, extramedullary expansion, and splenomegaly. Together with shortened red blood cell (RBC) survival, these abnormalities result in anemia. Patients with moderate or severe disease have increased intestinal iron absorption. Iron absorption, as well as iron recycling, is regulated by hepcidin; its binding to ferroportin (FPN-1) prevents iron egress from cells. Despite parenchymal iron overload in patients with β-thalassemia, hepcidin levels are low and do not appropriately increase in transfused patients with this disease. Relatively low levels of hepcidin mRNA expression in the liver are also characteristic of mouse models of β-thalassemia. This lack of an appropriate increase in hepcidin in β-thalassemia suggests that a competing signal is counter-regulating hepcidin expression despite increased parenchymal iron stores.

While phlebotomy, anemia, hypoxia, and stimulation with erythropoietin lead to the suppression of hepcidin, in the absence of erythropoiesis, hepcidin suppression does not occur. Furthermore, hepcidin expression decreases in vitro when hepatocytes are exposed to sera from β-thalassemia patients as compared to control sera and increases when exposed to sera from recently transfused β-thalassemia patients as compared to sera from the same patients just prior to transfusion. In light of the central role hepcidin plays in iron metabolism, the lack of an appropriate increase in hepcidin expression suggests that a paradoxical state of iron deficient erythropoiesis, despite increased parenchymal iron stores, exists in β-thalassemia. Hbb th1/th1 mice, the most commonly used murine model of β-thalassemia intermedia, when treated with iron, have increased hemoglobin production resulting from an expansion of extramedullary erythropoiesis.

Transferrin functions as the main transporter of iron in the circulation where it exists in an iron-free apo-transferrin (apoTf) form, as monoferric transferrin (monoTf), or as diferric holo-transferrin (holoTf). Typically, iron is bound to 30% of all transferrin binding sites in circulation. Transferrin-bound iron uptake by the transferrin receptor, transferrin receptor 1 (TfR1), is the only known means of iron delivery for erythropoiesis. The effect of transferrin on erythropoietic iron delivery is greater than stoichiometric as the transfer of iron to cells results in repeated recycling of transferrin and the conversion of holoTf to apoTf for further iron binding and transport in circulation. The inability to compensate for the ineffective erythropoiesis and anemia observed in β-thalassemia is, in part, a consequence of an insufficient amount of circulating transferrin. Although transferrin expression is regulated by several factors, normal levels of transferrin are insufficient to accommodate the tremendous expansion of erythropoiesis and alteration in iron stores in β-thalassemia.

The current standard of care for treating diseases associated with inefficient erythropoiesis include red blood cell transfusions and iron chelation therapy. However, there are many downsides that accompany red blood cell transfusions, such as the risk of infection, development of red blood cell antibodies, iron overload, splenomegaly, gastrointestinal effects, and cost, as well as problems with patient compliance with respect to iron chelation therapy.

### SUMMARY

The present disclosure is based, at least in part, on the use of monoferric transferrin to increase hepcidin expression, improve erythroid differentiation in diseases of ineffective erythropoiesis, and increase the relative concentration of monoferric transferrin relative to holotransferrin in the blood.

Accordingly, in one embodiment, provided herein are methods for increasing hepcidin expression, ameliorating ineffective erythropoiesis, and treating diseases or disorders associated with insufficient hepcidin expression or ineffective erythropoiesis in a subject in need thereof (e.g., a human subject or a non-human animal such as an Hbb th1/th1 mouse or an Hbb th3/+ mouse), comprising administering to said subject a therapeutically effective amount of a modified iron binding/releasing transferrin (MI-Tf). In one embodiment, the MI-Tf comprises a first lobe and a second lobe wherein each lobe independently binds, or is incapable of binding, and/or releases, or is incapable of releasing, iron. In a further embodiment, the first lobe binds iron, and the second lobe has a decreased binding affinity for iron. In another embodiment, both lobes bind iron, and the first lobe has a decreased ability to release iron. In another embodiment, the first lobe binds iron and has a decreased ability to release iron, and the second lobe has a decreased affinity for iron. In one embodiment, the methods result in at least one effect in said subject such as increased hepcidin expression, decreased non-transferrin bound iron (NTBI), reduced spleen size, decreased iron uptake by erythroid cells, increased erythroferrone, or decreased medullary and/or extramedullary erythropoiesis.

In one embodiment, the MI-Tf used in the methods herein is a human transferrin comprising at least one amino acid insertion, deletion, or substitution resulting in an altered ability to bind, and/or release, iron from at least one lobe of the transferrin. In another embodiment, one lobe of the MI-Tf does not, or cannot, bind iron. In another embodiment, the lobe with a decreased binding affinity for iron comprises at least one amino acid mutation that decreases the affinity of said lobe for iron. In another embodiment, the mutation reduces a negative charge within the iron binding cleft of said lobe.

In a further embodiment, the MI-Tf used in the methods herein comprises at least one amino acid substitution in a human transferrin (e.g., SEQ ID NO:2 or 3) at a position selected from Y95 (e.g., Y95F), Y188 (e.g., Y199F), Y426 (e.g., Y426F), Y517 (e.g., Y517F), D63 (e.g., D63S or D63C), G65 (e.g., G65R), R124 (e.g., R124E, R124S, R124A, or R124K), Y45 (e.g., Y45E), T120 (e.g., T120A), G394 (e.g., G394R), E357 (e.g., E357A), K511 (e.g., K511A), D356 (e.g., D356A), or H249 (e.g., H249Q). In further embodiments, the MI-Tf comprises amino acid substitutions at positions Y95 and Y188 (e.g., Y95F and Y188F) of the mature human transferrin amino acid sequence; or positions Y426 and Y517 (e.g., Y426F and Y517F) of the human transferrin amino acid sequence.

In another embodiment, the disease or disorder associated with insufficient hepcidin is associated with iron overload (e.g., non-transfusion-dependent iron overload or transfusion-dependent iron overload). In another embodiment, the disease or disorder associated with insufficient hepcidin is thalassemia, including α-thalassemia or β-thalassemia (e.g., transfusion-independent β-thalassemia). In another embodiment, the thalassemia is β-thalassemia intermedia, β-thalassemia major, hemoglobin E/β-thalassemia, or α-thalassemia intermedia (hemoglobin H disease). In another embodiment, the disease or disorder is hemochromatosis (e.g., hereditary hemochromatosis). In another embodiment, the disease or disorder is sickle cell anemia.

In another embodiment, the method comprises administering a course of a plurality of doses of the MI-Tf. In a further embodiment, the course comprises administering the MI-Tf for 7-21 days. In another embodiment, the course comprises administering at least one dose of the MI-Tf per day or at least one dose of the MI-Tf every other day. In another embodiment, the course comprises administering at least one dose of the MI-Tf per day for a predetermined number of days and at least one dose of the MI-Tf every other day for a predetermined number of days. In another embodiment the course is repeated at an interval selected the group consisting of: every other month, every third month, and every fourth month. In a further embodiment, the therapeutically effective amount comprises about 25-150 mg/kg of said MI-Tf In still a further embodiment, the MI-Tf is administered via a route selected from oral, parenteral, intravenous, intramuscular, subcutaneous, intranasal, transdermal, pulmonary, and rectal administration.

In another embodiment, the present disclosure provides a pharmaceutical composition comprising a therapeutically effective amount of an MI-Tf as described herein and a pharmaceutically acceptable carrier. In a further embodiment, the pharmaceutical composition is formulated for a route of administration selected from oral, parenteral, intravenous, intramuscular, subcutaneous, intranasal, transdermal, pulmonary, and rectal administration.

### BRIEF DESCRIPTION OF THE DRAWINGS

FIG. 1 depicts a urea-polyacrylamide gel electrophoresis of apotransferrin (apo-Tf) and di-ferric transferrin (dTf) samples incubated at 37° C. for 7 days in vitro at varying ratios of apo-TF and dTf.

FIG. 2 depicts the dose dependent decrease in cytosolic iron (P<0.001) and heme (P<0.01) in murine erythroleukemia (MEL) cells treated with escalating concentrations of apoTf, an effect abrogated by the additional of deferoxamine (DFO), an iron chelator. The urea gel of MEL cell supernatants revealed that monoferric Tf increased with increasing doses of apoTf and decreased in concurrently DFO-treated cells.

FIG. 3 depicts changes in β-globin mRNA expression in sorted bone marrow orthochromatophilic erythroblasts from apoTf- and PBS-treated th1/th1 thalassemic mice (“β-minor”). PBS-treated wild-type (WT) mice (“β-major”) were used as a control.

FIG. 4 depicts normalization of heme-regulated eIF2alpha kinase (HRI) and its target eIF2α (total and phosphorylated) expression in western blots of protein from sorted bone marrow erythroid precursors from apoTf− vs. PBS-treated th1/th1 mice.

### DETAILED DESCRIPTION OF THE DISCLOSURE

The present disclosure is based, at least in part, on the use of monoferric transferrin to increase hepcidin expression, improve erythroid differentiation in diseases of ineffective erythropoiesis, and increase the relative concentration of monoferric transferrin relative to holotransferrin in the blood.

The following definition of terms is provided as a helpful reference for the reader. The terms used in this patent have specific meanings as they related to the present disclosure. Every effort has been made to use terms according to their ordinary and common meaning. However, where a discrepancy exists between the common ordinary meaning and the following definitions, these definitions supersede common usage.

The term “administering” includes routes of administration which allow the protein composition to perform its intended function of increasing hepcidin expression, improving erythroid differentiation, and/or treating a disease or disorder disclosed herein. Depending on the route of administration, the composition can be coated with or disposed in a selected material to protect it from natural conditions which may detrimentally affect its ability to perform its intended function. The composition can be administered with other bioactive agents and/or with one or more pharmaceutically acceptable carriers. The composition can be administered prior to, during, or after the onset of symptoms of a disease or disorder disclosed herein, or prior to, during, or after the onset of a need for increasing hepcidin expression or improving erythroid differentiation.

The term “effective amount” or “therapeutically effective amount” of a modified iron binding/releasing transferrin is that amount necessary or sufficient to increase hepcidin expression, improve erythroid differentiation, or treating or prevent at least one symptom associated with a disease or disorder disclosed herein. The effective amount can vary depending on such factors as the size and weight of the subject, the type of illnesses, the severity of the symptoms or the particular composition used. One of ordinary skill in the art is able to study the aforementioned factors and make a determination regarding the effective amount of a composition without undue experimentation.

### Modified Iron Binding/Releasing Transferrins

Iron is transported between sites of acquisition, storage, and utilization by serum transferrin (also referred to herein as “Tf”). Tf is predominantly synthesized by the liver; its main role is to deliver iron to cells by receptor-mediated endocytosis. Tf circulates in three forms: diferric-Tf (“di-Tf”, “dTf”, “holotransferrin”, or “holo-Tf”; bound to two iron molecules), monoferric-Tf (“mono-Tf” or “mTf”; bound to one iron molecule), and apotransferrin (“apo-Tf”; unbound to iron) depending on available iron. The iron molecule can be located on either, or both of, an N- or C-terminal binding site (present in an N- or C-terminal lobe, respectively), as discussed herein.

As used herein the term “modified iron binding/releasing transferrin” (also referred to herein as “MI-Tf”) refers to a transferrin that is capable of binding one or two iron molecules, but is only capable of releasing one iron molecule (for example, when the transferrin binds to the transferrin receptor and delivers its bound iron to the cell). In some embodiments, an MI-Tf binds only one iron molecule. In other embodiments, an MI-Tf may bind two iron molecules, but can only release one of the iron molecules, while the other iron molecule remains bound to the transferrin, even under conditions where iron would be released from a wild-type transferrin.

The MI-Tf for use in the methods and compositions herein may have a reduced capacity to bind or release iron. In one embodiment, an MI-Tf has two lobes, an N-lobe and a C-lobe, wherein one lobe binds iron (e.g., binds iron with wild-type affinity), and wherein the other lobe has a reduced affinity for iron. In another embodiment, both lobes bind iron, but one lobe has a reduced ability to release iron. In another embodiment, one lobe binds iron but has a reduced ability to release iron, while the other lobe has a reduced affinity for iron. As used herein, the two lobes may be referred to as a “first lobe” and a “second lobe”. Either the N-lobe or the C-lobe may comprise the first lobe or the second lobe.

In another embodiment, the lobe with a reduced affinity for iron does not bind iron. In some embodiments, an MI-Tf as used herein may be referred to as “blocked transferrin” (“blocked Tf”; “bTf”), wherein blocked transferrin refers to a transferrin that is capable of binding to only one iron molecule. When bound to iron, blocked transferrin may thus be referred to as “monoferric transferrin (“monoTf”; “mTf”). In another embodiment, a blocked lobe may refer to a single transferrin lobe (N-lobe or C-lobe) that does not bind iron. Thus, a blocked transferrin may include one wild-type lobe and one blocked lobe. In another embodiment, the MI-Tf is capable of binding one and only one molecule of iron.

In a further embodiment, the MI-Tf retains affinity for the transferrin receptor TfR1 and retains the ability to induce intracellular signaling via TfR1.

In another embodiment, an MI-Tf as used herein by may be referred to as “locked transferrin” (“locked TF”; “ITf”), wherein locked transferrin refers to a transferrin wherein one lobe of said transferrin has a reduced ability to release its bound iron molecule. In another embodiment, a locked lobe may refer to a single transferrin lobe (N-lobe or C-lobe that cannot release its bound iron molecule. Thus a locked transferrin may include one wild-type lobe and one locked lobe, two locked lobes, or one locked lobe and one blocked lobe.

In another embodiment, an MI-Tf as used herein may comprise one wild-type lobe and one locked lobe. In another embodiment, an MI-Tf as used herein may comprise one wild-type lobe and one blocked lobe. In another embodiment, an MI-Tf as used herein may comprise one blocked lobe and one locked lobe.

In further embodiments, as used herein, a diferric locked transferrin is bound to two iron molecules and is incapable of releasing any iron. A diferric hemi-locked transferrin is bound to two iron molecules and is capable of releasing one iron molecule. A monoferric hemi-blocked transferrin is bound to one iron molecule and capable of releasing one iron molecule. A monoferric blocked/locked transferrin is bound to one iron molecule and incapable of releasing any iron. In other embodiments, a diferric wild-type transferrin polypeptide is bound to two iron molecules and is capable of releasing two iron molecules to a cell. A fully blocked transferrin may comprise two blocked lobes, resulting in an apotransferrin incapable of binding any iron. A fully locked transferrin may comprise two locked lobes, resulting in a transferrin polypeptide incapable of releasing any iron. Wild-type, fully blocked, and fully locked transferrin may be useful, for example, as positive or negative controls in experimental studies, or as supplemental therapeutic agents in combination with the MI-Tf disclosed herein.

In a further embodiment, the MI-Tf are not bound to any iron prior to incorporation into a pharmaceutical composition and/or prior to administration to a subject. Such transferrin compositions may be referred to herein as “iron free”.

In another embodiment, the MI-Tf are modified human transferrin polypeptides, e.g., a human transferrin polypeptide modified by a deletion, insertion, or substitution of at least one amino acid, such that the modified transferrin is an MI-Tf with a reduced affinity for iron in one lobe and/or a reduced ability to release iron from one lobe. In another embodiment, an MI-Tf may be modified via chemical means, such that the chemical modification results in one lobe with a reduced affinity for iron and/or one lobe with a reduced ability to release iron.

In other embodiments, the MI-Tf may be derived from transferrins from other species, e.g., mouse, rat, sheep, goat, cow, horse, cat, dog, rabbit, chicken, or monkey or other non-human primate.

Modified iron binding/releasing transferrins useful in the methods and compositions disclosed herein may be produced by any known method for producing polypeptides, particularly therapeutic polypeptides. Recombinant production methods are well-known to those of skill in the art, as is chemical synthesis. For example, MI-Tf may be produced in any known protein production system, including prokaryotic (e.g., bacteria such as E. coli) or eukaryotic systems, including, but not limited to, yeast (e.g., S. cerevisiae), algae, plants (e.g., rice or tobacco), insect cells (e.g., using a baculovirus expression system), or mammalian cells (e.g., COS or CHO cells). Methods for modulating post-translational modification may also be used. For example, the glycosylation pattern of an MI-Tf may be modified through the choice of host cells used for polypeptide expression and/or through the introduction of amino acid substitutions that increase or decrease glycosylation. See, for example, US 2012/0088729, incorporated herein by reference for all it discloses regarding production of non-glycosylated transferrin in plants. See also U.S. Pat. No. 6,825,037, incorporated herein by reference for all it contains regarding transferrin mutants with reduced glycosylation, as well as for methods for producing recombinant transferrin polypeptides. Transferrin proteins may also be isolated from subjects and modified, e.g., via chemical means, in order to produce an MI-Tf. In one embodiment, transferrin is isolated from human plasma Cohn Fraction IV, e.g., as a byproduct of human plasma fractionation used for production of other plasma based products, and then modified to produce an MI-Tf.

The human transferrin mRNA sequence is disclosed in GenBank Accession No. NM_001063 and is set forth as SEQ ID NO:1. Nucleotides 309-2405 of SEQ ID NO:1 comprise the open reading frame encoding the transferrin precursor polypeptide sequence.

The full-length human transferrin precursor polypeptide sequence is disclosed in GenBank Accession No. NP_001054 and is shown below (set forth as SEQ ID NO:2). Amino acids 1-19 of SEQ ID NO:2 correspond to the predicted signal peptide and are indicated by a double underline. Amino acids 20-698 of SEQ ID NO:2 correspond to the mature transferrin sequence and are further set forth as SEQ ID NO:3 and indicated in bold in SEQ ID NO:2 below. Non-limiting examples of amino acids that may be modified in the MI-Tf proteins in certain embodiments described herein are indicated by a single underline.

(SEQ ID NO: 2)
MRLAVGALLVCAVLGLCLAVPDKTVRWCAVSEHEATKCQSFRDHMKS
VIPSDGPSVACVKKASYLDCIRAIAANEADAVTLDAGLVYDAYLAPN
NLKPVVAEFYGSKEDPQTFYYAVAVVKKDSGFQMNQLRGKKSCHTGL
GRSAGWNIPIGLLYCDLPEPRKPLEKAVANFFSGSCAPCADGTDFPQ
LCQLCPGCGCSTLNQYFGYSGAFKCLKDGAGDVAFVKHSTIFENLAN
KADRDQYELLCLDNTRKPVDEYKDCHLAQVPSHTVVARSMGGKEDLI
WELLNQAQEHEGKDKSKEFQLFSSPHGKDLLEKDSAHGELKVPPRMD
AKMYLGYEYVTAIRNLREGTCPEAPTDECKPVKWCALSHHERLKCDE
WSVNSVGKIECVSAETTEDCIAKIMNGEADAMSLDGGFVYIAGKCGL
VPVLAENYNKSDNCEDTPEAGYFAVAVVKKSASDLTWDNLKGKKSCH
TAVGRTAGWNIPMGLLYNKINHCRFDEFFSEGCAPGSKKDSSLCKLC
MGSGLNLCEPNNKEGYYGYTGAFRCLVEKGDVAFVKHQTVPQNTGGK
NPDPWAKNLNEKDYELLCLDGTRKPVEEYANCHLARAPNHAVVTRKD
KEACVHKILRQQQHLFGSNVTDCSGNFCLFRSETKDLLFRDDTVCLA
KLHDRNTYEKYLGEEYVKAVGNLRKCSTSSLLEACTFRRP

In another embodiment, an MI-Tf is based on a mature transferrin sequence (i.e., a transferrin polypeptide sequence wherein the signal peptide has been removed). In one embodiment, the MI-Tf comprises at least one amino acid substitution, deletion, or insertion, as compared to a wild-type transferrin polypeptide sequence. In another embodiment, the MI-Tf comprises at least one amino acid substitution, deletion, or insertion, as compared to a wild-type human transferrin polypeptide sequence (e.g., the human transferrin polypeptide sequence of SEQ ID NO:2 or SEQ ID NO:3). In another embodiment, the MI-Tf comprises at least one amino acid substitution, deletion, or insertion, such that the MI-Tf amino acid sequence is at least 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to the human transferrin amino acid sequence of SEQ ID NO:2 or SEQ ID NO:3, wherein the MI-Tf comprises one lobe that binds iron and one lobe with a reduced affinity for iron. In another embodiment, the MI-Tf comprises at least 2, 3, 4, 5, 6, 7, 8, 9, 10, 11, 12, 13, or 14 amino acid substitutions, deletions, and/or insertions as compared to a wild-type human transferrin polypeptide sequence (e.g., the human transferrin polypeptide sequence of SEQ ID NO:2 or SEQ ID NO:3).

In another embodiment, the MI-Tf comprises at least one amino acid substitution at a position corresponding to Y95, Y188, Y426, Y517, D63, G65, R124, Y45, T120, G394, E357, K511, D356, or H249 of the mature human transferrin amino acid sequence. The amino acid positions are identified by the amino acid letter code and the position of that amino acid in the sequence. For example, Y95 refers to the tyrosine residue (Y) at position 95 of the mature human transferrin amino acid sequence (i.e., SEQ ID NO:3). In another embodiment the MI-Tf is a non-human transferrin sequence, wherein said non-human transferrin sequence comprises at least one amino acid substitution at a position homologous to a position corresponding to Y95, Y188, Y426, Y517, D63, G65, R124, Y45, T120, G394, E357, K511, D356, or H249 in the mature human transferrin amino acid sequence. The sequences of non-human transferrin proteins are known in the art, as are methods for aligning amino acid sequences and identifying homologous amino acids from different species.

In another embodiment, the MI-Tf comprises at least one substitution selected from the group consisting of: Y95F, Y188F, Y426F, Y517F, D63S, D63C, G65R, R124E, R124S, R124A, R124K, Y45E, T120A, G394R, E357A, K511A, D356A, and H249Q in the mature human transferrin amino acid sequence. The substituted positions are identified by the amino acid letter code and the position of that amino acid in the sequence, followed by the substitute amino acid letter code. For example, Y95F refers to a substitution at position 95 from tyrosine (Y) to phenylalanine (F).

In one embodiment, the MI-Tf comprises amino acid substitutions at positions Y95 and Y188 of the mature human transferrin amino acid sequence. In a further embodiment, said substitutions comprise Y95F and Y188F.

In another embodiment, the MI-Tf comprises amino acid substitutions at positions Y426 and Y517 of the mature human transferrin amino acid sequence. In a further embodiment, said substitutions comprise Y426F and Y517F.

In another embodiment, the MI-Tf comprises an amino acid substitution at position D63 of the mature human transferrin amino acid sequence. In a further embodiment, said substitution is selected from D63S and D63C.

In another embodiment, the MI-Tf comprises an amino acid substitution at position G65 of the mature human transferrin amino acid sequence. In a further embodiment, said substitution comprises G65R.

In another embodiment, the MI-Tf comprises an amino acid substitution at position R124 of the mature human transferrin amino acid sequence. In a further embodiment, said substitution is selected from R124E, R124S, R124A, and R124K.

In another embodiment, the MI-Tf comprises an amino acid substitution at position Y45 of the mature human transferrin amino acid sequence. In a further embodiment, said substitution comprises Y45E.

In another embodiment, the MI-Tf comprises an amino acid substitution at position T120 of the mature human transferrin amino acid sequence. In a further embodiment, said substitution comprises T120A.

In another embodiment, the MI-Tf comprises an amino acid substitution at position G394 of the mature human transferrin amino acid sequence. In a further embodiment, said substitution comprises G394R.

In another embodiment, the MI-Tf comprises an amino acid substitution at position E357 of the mature human transferrin amino acid sequence. In a further embodiment, said substitution comprises E357A.

In another embodiment, the MI-Tf comprises an amino acid substitution at position K511 of the mature human transferrin amino acid sequence. In a further embodiment, said substitution comprises K511A.

In another embodiment, the MI-Tf comprises an amino acid substitution at position D356 of the mature human transferrin amino acid sequence. In a further embodiment, said substitution comprises D356A.

In another embodiment, the MI-Tf comprises an amino acid substitution at position H249 of the mature human transferrin amino acid sequence. In a further embodiment, said substitution comprises H249Q.

Amino acid residues important for iron binding, as well as MI-Tf with a reduced affinity for iron in one lobe, including those with specific substitutions described above, are described in further detail in: Mason et al., (2004) Protein Expr. Purif. 36:318-326; Grady et al., (1995) Biochem. J. 309:403-410; Adams et al., (2002) J. Biol. Chem. 278:6027-6033; Mason et al., (2009) Biochemistry 48(9):1945-1953; Eckenroth et al., (2011) Proc. Natl. Acad. Sci. USA. 108(32):13089-13094; and Steere et al., (2012) Biochemistry. 51(2):686-694.

Additional MI-Tf with reduced affinity for iron in one lobe can be identified by one of skill in the art, for example, by modifying a transferrin polypeptide, including any of the transferrin polypeptides described herein, e.g., by introducing at least one amino acid substitution, deletion, or insertion, or by introducing at least one post-translational modification. These modified transferrin polypeptides can then be tested for their ability to bind iron using methods known in the art. In one embodiment, modified transferrin polypeptides can be produced using site-directed mutagenesis (e.g., mutagenesis targeting specific amino acid residues of transferrin known to be or suspected of being involved in iron binding). In another embodiment, modified transferrin polypeptides can be produced in a transferrin peptide library comprising unknown or random mutations. Methods for determining the ability of a modified transferrin polypeptide to bind iron are known in the art and are disclosed, for example, in Mason et al. 2004. For example, in one embodiment, the ability of a modified transferrin to bind iron can be determined using gel electrophoresis (e.g., a Novex™ 6% TBE-urea mini-gel in 90 mM Tris-borate, pH 8.4, containing 16 mM EDTA). Apo-transferrin, monoferric transferrin with iron in the N-lobe, monoferric transferrin with iron in the C-lobe, and diferric transferrin migrate at different rates; transferrin proteins with known iron-binding capacities can thus be used as standards to identify MI-Tfs with unknown iron-binding capacities.

### Pharmaceutical Compositions

Aspects of the present disclosure provide, in part, a pharmaceutical composition comprising an MI-Tf. An MI-Tf includes the compounds disclosed herein. The compositions disclosed herein may, or may not, comprise any number and combination of compounds disclosed herein. For instance, a composition can comprise, e.g., two or more MI-Tf disclosed herein, three or more MI-Tf disclosed herein, four or more MI-Tf disclosed herein, or five or more MI-Tf disclosed herein. The pharmaceutical compositions may further comprise one or more additional therapeutic agents, e.g., one or more additional therapeutic agents useful for increasing hepcidin expression, modulating ineffective erythropoiesis, or treating a disease or disorder as disclosed herein.

An MI-Tf disclosed herein, or a composition comprising such an MI-Tf, is generally administered to an individual as a pharmaceutical composition. Pharmaceutical compositions may be prepared by combining a therapeutically effective amount of at least one MI-Tf as disclosed herein, or a pharmaceutically acceptable acid addition salt thereof, as an active ingredient, with conventional acceptable pharmaceutical excipients, and by preparation of unit dosage forms suitable for therapeutic use. As used herein, the term “pharmaceutical composition” and refers to a therapeutically effective concentration of an active compound, such as, e.g., any of the MI-Tf disclosed herein. Preferably, the pharmaceutical composition does not produce an adverse, allergic, or other untoward or unwanted reaction when administered to an individual. A pharmaceutical composition disclosed herein is useful for medical and veterinary applications. A pharmaceutical composition may be administered to an individual alone, or in combination with other supplementary active compounds, agents, drugs or hormones. The pharmaceutical compositions may be manufactured using any of a variety of processes, including, without limitation, conventional mixing, dissolving, granulating, dragee-making, levigating, emulsifying, encapsulating, entrapping, and lyophilizing. The pharmaceutical composition can take any of a variety of forms including, without limitation, a sterile solution, suspension, emulsion, lyophilizate, tablet, pill, pellet, capsule, powder, syrup, elixir, or any other dosage form suitable for administration.

The disclosed compositions may be formulated for any desirable route of delivery including, but not limited to, parenteral, intravenous, intradermal, subcutaneous, oral, transdermal, transmucosal, rectal, intraperitoneal, intranasal, pulmonary, and buccal.

A pharmaceutical composition produced using the methods disclosed herein may be a liquid formulation, semi-solid formulation, or a solid formulation. A formulation disclosed herein can be produced in a manner to form one phase, such as, e.g., an oil or a solid. Alternatively, a formulation disclosed herein can be produced in a manner to form two phase, such as, e.g., an emulsion. A pharmaceutical composition disclosed herein intended for such administration may be prepared according to any method known to the art for the manufacture of pharmaceutical compositions.

Liquid formulations suitable for parenteral injection may comprise physiologically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethyleneglycol (PEG), glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

In certain aspects, parenteral, intradermal or subcutaneous formulations may be sterile injectable aqueous or oleaginous suspensions. Acceptable vehicles, solutions, suspensions and solvents may include, but are not limited to, water or other sterile diluent; saline; Ringer's solution; sodium chloride; fixed oils such as mono- or diglycerides; fatty acids such as oleic acid; polyethylene glycols; glycerin; propylene glycol or other synthetic solvents; antibacterial agents such as benzyl alcohol; antioxidants such as ascorbic acid; chelating agents such as ethylenediaminetetraacetic acid; buffers such as acetates, citrates or phosphates; and agents for the adjustment of tonicity such as sodium chloride or dextrose.

Solutions or suspensions used for parenteral, intradermal, or subcutaneous application may include one or more of the following components: a sterile diluent such as water for injection, saline solution, fixed oils, polyethylene glycols, glycerin; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation may be enclosed in ampoules, disposable syringes or multiple dose vials made of glass or plastic.

Pharmaceutical compositions suitable for injectable use may include sterile aqueous solutions or dispersions and sterile powders for the extemporaneous preparation of sterile injectable solutions or dispersion. For intravenous administration, suitable carriers include, but are not limited to, saline, bacteriostatic water, CREMOPHOR EL® (BASF, Parsippany, N.J.) or phosphate buffered saline (PBS). The solvent or dispersion medium may contain, for example, water, ethanol, a polyol (for example, glycerol, propylene glycol, and liquid polyetheylene glycol, and the like), and suitable mixtures thereof. Proper fluidity can be maintained, for example, by the use of a coating such as lecithin, by the maintenance of the requited particle size in the case of dispersion and by the use of surfactants. Preventing growth of microorganisms can be achieved by various antibacterial and antifungal agents, for example, parabens, chlorobutanol, phenol, ascorbic acid, thimerosal, and the like. The composition may also include isotonic agents such as, for example, sugars; polyalcohols such as mannitol; sorbitol; or sodium chloride. Prolonged absorption of injectable compositions can be enhanced by addition of an agent which delays absorption, such as, for example, aluminum monostearate or gelatin.

Systemic administration may be by transmucosal or transdermal means. For transmucosal or transdermal administration, penetrants may be used. Such penetrants are generally known in the art, and include, for example, detergents, bile salts, and fusidic acid derivatives. Transdermal administration may include a bioactive agent and may be formulated into ointments, salves, gels, or creams as generally known in the art. Transmucosal administration may be accomplished through the use of nasal sprays or suppositories.

Semi-solid formulations suitable for topical administration include, without limitation, ointments, creams, salves, and gels. In such solid formulations, the MI-Tf may be admixed with at least one inert customary excipient (or carrier) such as, a lipid and/or polyethylene glycol.

Solid formulations suitable for oral administration include capsules, tablets, pills, powders and granules. In such solid formulations, the MI-Tf may be admixed with at least one inert customary excipient (or carrier) such as sodium citrate or dicalcium phosphate or (a) fillers or extenders, as for example, starches, lactose, sucrose, glucose, mannitol and silicic acid, (b) binders, as for example, carboxymethylcellulose, alignates, gelatin, polyvinylpyrrolidone, sucrose and acacia, (c) humectants, as for example, glycerol, (d) disintegrating agents, as for example, agar-agar, calcium carbonate, potato or tapioca starch, alginic acid, certain complex silicates and sodium carbonate, (e) solution retarders, as for example, paraffin, (f) absorption accelerators, as for example, quaternary ammonium compounds, (g) wetting agents, as for example, cetyl alcohol and glycerol monostearate, (h) adsorbents, as for example, kaolin and bentonite, and (i) lubricants, as for example, talc, calcium stearate, magnesium stearate, solid polyethylene glycols, sodium lauryl sulfate or mixtures thereof. In the case of capsules, tablets and pills, the dosage forms may also comprise buffering agents.

In liquid and semi-solid formulations, a concentration of an MI-Tf disclosed herein typically may be between about 0.01 mg/mL to about 1,000 mg/mL. In another embodiment, the concentration may be 0.03 mg/mL to about 0.05 mg/mL. In aspects of this embodiment, a therapeutically effective amount of a therapeutic compound disclosed herein may be from, e.g., about 50 mg/mL to about 100 mg/mL, about 50 mg/mL to about 200 mg/mL, about 50 mg/mL to about 300 mg/mL, about 50 mg/mL to about 400 mg/mL, about 50 mg/mL to about 500 mg/mL, about 50 mg/mL to about 600 mg/mL, about 50 mg/mL to about 700 mg/mL, about 50 mg/mL to about 800 mg/mL, about 50 mg/mL to about 900 mg/mL, about 50 mg/mL to about 1,000 mg/mL, about 100 mg/mL to about 200 mg/mL, about 100 mg/mL to about 300 mg/mL, about 100 mg/mL to about 400 mg/mL, about 100 mg/mL to about 500 mg/mL, about 100 mg/mL to about 600 mg/mL, about 100 mg/mL to about 700 mg/mL, about 100 mg/mL to about 800 mg/mL, about 100 mg/mL to about 900 mg/mL, about 100 mg/mL to about 1,000 mg/mL, about 200 mg/mL to about 300 mg/mL, about 200 mg/mL to about 400 mg/mL, about 200 mg/mL to about 500 mg/mL, about 200 mg/mL to about 600 mg/mL, about 200 mg/mL to about 700 mg/mL, about 200 mg/mL to about 800 mg/mL, about 200 mg/mL to about 900 mg/mL, about 200 mg/mL to about 1,000 mg/mL, about 300 mg/mL to about 400 mg/mL, about 300 mg/mL to about 500 mg/mL, about 300 mg/mL to about 600 mg/mL, about 300 mg/mL to about 700 mg/mL, about 300 mg/mL to about 800 mg/mL, about 300 mg/mL to about 900 mg/mL, about 300 mg/mL to about 1,000 mg/mL, about 400 mg/mL to about 500 mg/mL, about 400 mg/mL to about 600 mg/mL, about 400 mg/mL to about 700 mg/mL, about 400 mg/mL to about 800 mg/mL, about 400 mg/mL to about 900 mg/mL, about 400 mg/mL to about 1,000 mg/mL, about 500 mg/mL to about 600 mg/mL, about 500 mg/mL to about 700 mg/mL, about 500 mg/mL to about 800 mg/mL, about 500 mg/mL to about 900 mg/mL, about 500 mg/mL to about 1,000 mg/mL, about 600 mg/mL to about 700 mg/mL, about 600 mg/mL to about 800 mg/mL, about 600 mg/mL to about 900 mg/mL, or about 600 mg/mL to about 1,000 mg/mL.

In semi-solid and solid formulations, an amount of an MI-Tf disclosed herein typically may be between about 0.001% to about 45% by weight. In aspects of this embodiment, an amount of a therapeutic compound disclosed herein may be from, e.g., about 0.1% to about 45% by weight, about 0.1% to about 40% by weight, about 0.1% to about 35% by weight, about 0.1% to about 30% by weight, about 0.1% to about 25% by weight, about 0.1% to about 20% by weight, about 0.1% to about 15% by weight, about 0.1% to about 10% by weight, about 0.1% to about 5% by weight, about 1% to about 45% by weight, about 1% to about 40% by weight, about 1% to about 35% by weight, about 1% to about 30% by weight, about 1% to about 25% by weight, about 1% to about 20% by weight, about 1% to about 15% by weight, about 1% to about 10% by weight, about 1% to about 5% by weight, about 5% to about 45% by weight, about 5% to about 40% by weight, about 5% to about 35% by weight, about 5% to about 30% by weight, about 5% to about 25% by weight, about 5% to about 20% by weight, about 5% to about 15% by weight, about 5% to about 10% by weight, about 10% to about 45% by weight, about 10% to about 40% by weight, about 10% to about 35% by weight, about 10% to about 30% by weight, about 10% to about 25% by weight, about 10% to about 20% by weight, about 10% to about 15% by weight, about 15% to about 45% by weight, about 15% to about 40% by weight, about 15% to about 35% by weight, about 15% to about 30% by weight, about 15% to about 25% by weight, about 15% to about 20% by weight, about 20% to about 45% by weight, about 20% to about 40% by weight, about 20% to about 35% by weight, about 20% to about 30% by weight, about 20% to about 25% by weight, about 25% to about 45% by weight, about 25% to about 40% by weight, about 25% to about 35% by weight, or about 25% to about 30% by weight.

A pharmaceutical composition disclosed herein can optionally include a pharmaceutically acceptable carrier that facilitates processing of an MI-Tf into pharmaceutically acceptable compositions. As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. As used herein, the term “pharmacologically acceptable carrier” is synonymous with “pharmacological carrier” and refers to any carrier that has substantially no long term or permanent detrimental effect when administered and encompasses terms such as “pharmacologically acceptable vehicle, stabilizer, diluent, additive, auxiliary, or excipient.” Such a carrier generally is mixed with an MI-Tf or permitted to dilute or enclose the MI-Tf and can be a solid, semi-solid, or liquid agent. It is understood that the MI-Tf can be soluble or can be delivered as a suspension in the desired carrier or diluent. Any of a variety of pharmaceutically acceptable carriers can be used including, without limitation, aqueous media such as, e.g., water, saline, glycine, hyaluronic acid and the like; solid carriers such as, e.g., starch, magnesium stearate, mannitol, sodium saccharin, talcum, cellulose, glucose, sucrose, lactose, trehalose, magnesium carbonate, and the like; solvents; dispersion media; coatings; antibacterial and antifungal agents; isotonic and absorption delaying agents; or any other inactive ingredient. Selection of a pharmacologically acceptable carrier can depend on the mode of administration. Except insofar as any pharmacologically acceptable carrier is incompatible with the MI-Tf, its use in pharmaceutically acceptable compositions is contemplated. Non-limiting examples of specific uses of such pharmaceutical carriers can be found in Pharmaceutical Dosage Forms and Drug Delivery Systems (Howard C. Ansel et al., eds., Lippincott Williams & Wilkins Publishers, 7th ed. 1999); Remington: The Science and Practice of Pharmacy (Alfonso R. Gennaro ed., Lippincott, Williams & Wilkins, 20th ed. 2000); Goodman & Gilman's The Pharmacological Basis of Therapeutics (Joel G. Hardman et al., eds., McGraw-Hill Professional, 10th ed. 2001); and Handbook of Pharmaceutical Excipients (Raymond C. Rowe et al., APhA Publications, 4th edition 2003). These protocols are routine and any modifications are well within the scope of one skilled in the art and from the teaching herein.

A pharmaceutical composition disclosed herein can optionally include, without limitation, other pharmaceutically acceptable components (or pharmaceutical components), including, without limitation, buffers, preservatives, tonicity adjusters, salts, antioxidants, osmolality adjusting agents, physiological substances, pharmacological substances, bulking agents, emulsifying agents, wetting agents, sweetening or flavoring agents, and the like. Various buffers and means for adjusting pH can be used to prepare a pharmaceutical composition disclosed herein, provided that the resulting preparation is pharmaceutically acceptable. Such buffers include, without limitation, acetate buffers, borate buffers, citrate buffers, phosphate buffers, neutral buffered saline, and phosphate buffered saline. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole, and butylated hydroxytoluene. Useful preservatives include, without limitation, benzalkonium chloride, chlorobutanol, thimerosal, phenylmercuric acetate, phenylmercuric nitrate, a stabilized oxy chloro composition, such as, e.g., sodium chlorite and chelants, such as, e.g., DTPA or DTPA-bisamide, calcium DTPA, and CaNaDTPA-bisamide. Tonicity adjustors useful in a pharmaceutical composition include, without limitation, salts such as, e.g., sodium chloride, potassium chloride, mannitol or glycerin and other pharmaceutically acceptable tonicity adjustor. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. It is understood that these and other substances known in the art of pharmacology can be included in a pharmaceutical composition useful in the methods disclosed herein.

An MI-Tf disclosed herein, or a composition comprising such an MI-Tf, may also be incorporated into a drug delivery platform in order to achieve a controlled release profile over time. Such a drug delivery platform comprises an MI-Tf disclosed herein dispersed within a polymer matrix, typically a biodegradable, bioerodible, and/or bioresorbable polymer matrix. As used herein, the term “polymer” refers to synthetic homo- or copolymers, naturally occurring homo- or copolymers, as well as synthetic modifications or derivatives thereof having a linear, branched or star structure. Copolymers can be arranged in any form, such as, e.g., random, block, segmented, tapered blocks, graft, or triblock. Polymers are generally condensation polymers. Polymers can be further modified to enhance their mechanical or degradation properties by introducing cross-linking agents or changing the hydrophobicity of the side residues. If crosslinked, polymers are usually less than 5% crosslinked, usually less than 1% crosslinked.

Suitable polymers include, without limitation, alginates, aliphatic polyesters, polyalkylene oxalates, polyamides, polyamidoesters, polyanhydrides, polycarbonates, polyesters, polyethylene glycol, polyhydroxyaliphatic carboxylic acids, polyorthoesters, polyoxaesters, polypeptides, polyphosphazenes, polysaccharides, and polyurethanes. The polymer usually comprises at least about 10% (w/w), at least about 20% (w/w), at least about 30% (w/w), at least about 40% (w/w), at least about 50% (w/w), at least about 60% (w/w), at least about 70% (w/w), at least about 80% (w/w), or at least about 90% (w/w) of the drug delivery platform. Examples of biodegradable, bioerodible, and/or bioresorbable polymers and methods useful to make a drug delivery platform are described in, e.g., U.S. Pat. No. 4,756,911; U.S. Pat. No. 5,378,475; U.S. Pat. No. 7,048,946; U.S. Patent Publication 2005/0181017; U.S. Patent Publication 2005/0244464; U.S. Patent Publication 2011/0008437; each of which is incorporated by reference for all they disclose regarding drug delivery compositions and methods.

In aspects of this embodiment, a polymer composing the matrix is a polypeptide such as, e.g., silk fibroin, keratin, or collagen. In other aspects of this embodiment, a polymer composing the matrix is a polysaccharide such as, e.g., cellulose, agarose, elastin, chitosan, chitin, or a glycosaminoglycan like chondroitin sulfate, dermatan sulfate, keratan sulfate, or hyaluronic acid. In yet other aspects of this embodiment, a polymer composing the matrix is a polyester such as, e.g., D-lactic acid, L-lactic acid, racemic lactic acid, glycolic acid, caprolactone, and combinations thereof.

One of ordinary skill in the art appreciates that the selection of a suitable polymer for forming a suitable disclosed drug delivery platform depends on several factors. The more relevant factors in the selection of the appropriate polymer(s), include, without limitation, compatibility of polymer with MI-Tf, desired release kinetics of MI-Tf, desired biodegradation kinetics of platform at implantation site, desired bioerodible kinetics of platform at implantation site, desired bioresorbable kinetics of platform at implantation site, in vivo mechanical performance of platform, processing temperatures, biocompatibility of platform, and patient tolerance. Other relevant factors that, to some extent, dictate the in vitro and in vivo behavior of the polymer include the chemical composition, spatial distribution of the constituents, the molecular weight of the polymer and the degree of crystallinity.

A drug delivery platform includes both a sustained release drug delivery platform and an extended release drug delivery platform. As used herein, the term “sustained release” refers to the release of an MI-Tf disclosed herein over a period of about seven days or more. As used herein, the term “extended release” refers to the release of an MI-Tf disclosed herein over a period of time of less than about seven days.

In aspects of this embodiment, a sustained release drug delivery platform releases an MI-Tf disclosed herein with substantially first order release kinetics over a period of, e.g., about 7 days after administration, about 15 days after administration, about 30 days after administration, about 45 days after administration, about 60 days after administration, about 75 days after administration, or about 90 days after administration. In other aspects of this embodiment, a sustained release drug delivery platform releases an MI-Tf disclosed herein with substantially first order release kinetics over a period of, e.g., at least 7 days after administration, at least 15 days after administration, at least 30 days after administration, at least 45 days after administration, at least 60 days after administration, at least 75 days after administration, or at least 90 days after administration.

In aspects of this embodiment, a drug delivery platform releases an MI-Tf disclosed herein with substantially first order release kinetics over a period of, e.g., about 1 day after administration, about 2 days after administration, about 3 days after administration, about 4 days after administration, about 5 days after administration, or about 6 days after administration. In other aspects of this embodiment, a drug delivery platform releases an MI-Tf disclosed herein with substantially first order release kinetics over a period of, e.g., at most 1 day after administration, at most 2 days after administration, at most 3 days after administration, at most 4 days after administration, at most 5 days after administration, or at most 6 days after administration.

In a one embodiment, the MI-Tf can easily be administered parenterally such as for example, by intravenous, intramuscular, or subcutaneous injection. Parenteral administration can be accomplished by incorporating the compounds into a solution or suspension. Such solutions or suspensions may also include sterile diluents such as water for injection, saline solution, fixed oils, polyethylene glycols, glycerin, propylene glycol or other synthetic solvents. Parenteral formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Rectal administration includes administering the MI-Tf, in a pharmaceutical composition, into the rectum or large intestine. This can be accomplished using suppositories or enemas. Suppository formulations can easily be made by methods known in the art. For example, suppository formulations can be prepared by heating glycerin to about 120° C., dissolving the composition in the glycerin, mixing the heated glycerin after which purified water may be added, and pouring the hot mixture into a suppository mold.

Transdermal administration includes percutaneous absorption of the composition through the skin. Transdermal formulations include patches (such as the well-known nicotine patch), ointments, creams, gels, salves and the like.

### Methods of Treatment

In one embodiment, disclosed herein are methods for increasing hepcidin expression, ameliorating ineffective erythropoiesis, and treating diseases or disorders associated with insufficient hepcidin expression or ineffective erythropoiesis in a subject in need thereof (e.g., a human subject or a non-human animal suffering from a disorder associated with ineffective erythropoiesis or insufficient hepcidin expression), comprising administering to the subject a therapeutically effective amount of an MI-Tf, wherein the MI-Tf comprises two lobes (e.g., an N-lobe and a C-lobe), and wherein one of the lobes binds iron, and wherein the other lobe has a decreased binding affinity for iron. In another embodiment, the methods result in at least one effect in the subject selected from the group consisting of: increased hepcidin expression, decreased non-transferrin bound iron (NTBI), reduced spleen size, decreased iron uptake by erythroid cells, increased erythroferrone, and decreased medullary and/or extramedullary erythropoiesis.

In another embodiment, the disease or disorder associated with insufficient hepcidin is associated with iron overload (e.g., non-transfusion-dependent iron overload or transfusion-dependent iron overload). In another embodiment, the disease or disorder associated with insufficient hepcidin is thalassemia, including α-thalassemia or β-thalassemia (e.g., transfusion-independent β-thalassemia). In another embodiment, the thalassemia is selected from the group consisting of: β-thalassemia intermedia, β-thalassemia major, hemoglobin E/β-thalassemia and α-thalassemia intermedia (hemoglobin H disease). In another embodiment, the disease or disorder is hemochromatosis (e.g., hereditary hemochromatosis). In another embodiment, the disease or disorder is a myelodysplastic syndrome. In another embodiment, the disease or disorder is sickle cell anemia.

In another embodiment, the method comprises administering a course of a plurality of doses of the MI-Tf. In a further embodiment, the course comprises administering the MI-Tf for 7-21 days. In another embodiment, the course comprises administering at least one dose of the MI-Tf per day or at least one dose of the MI-Tf every other day. In another embodiment, the course comprises administering at least one dose of the MI-Tf per day for a predetermined number of days and at least one dose of the MI-Tf every other day for a predetermined number of days. In another embodiment the course is repeated at an interval selected the group consisting of: every other month, every third month, and every fourth month. In a further embodiment, the therapeutically effective amount comprises about 25-150 mg/kg of the MI-Tf. In still a further embodiment, the MI-Tf is administered via a route including, but not limited to, oral, parenteral, intravenous, intramuscular, subcutaneous, intranasal, transdermal, pulmonary, and rectal administration.

The safety of human transferrin injections has already been demonstrated. The disclosed methods are useful for treatment of splenectomized or non-splenectomized subjects. In one embodiment, the subject has splenomegaly. In another embodiment, the subject has a history of splenomegaly and is splenectomized. In another embodiment, the subject has a history of splenomegaly but is not splenectomized.

The subject may be any subject that would benefit from an increase in hepcidin expression, including, but not limited to, a human, mouse, rat, monkey, horse, cow, bull, steer, sheep, goat, cat, dog, or chicken. In a one embodiment, the subject is a mammal. In a further embodiment, the subject is a livestock, veterinary, or companion animal. In a further embodiment, the subject is a primate. In a further embodiment, the subject is a human. In a further embodiment, the subject has a disease or disorder described herein. In another embodiment, the subject is a non-human animal model for a human disease or disorder described herein.

In accordance with the methods disclosed herein, the MI-Tf may be administered to a human or other animal subject by known procedures, including, without limitation, nasal administration, oral administration, parenteral administration (e.g., epifascial, intracapsular, intracutaneous, intradermal, intramuscular, intraorbital, intraperitoneal, intrasternal, intravascular, intravenous, parenchymatous, and subcutaneous administration), sublingual administration, transdermal administration, and administration by osmotic pump.

In one embodiment, a MI-Tf is administered by intravenous infusion over a period of time, such as from 15 minutes to 2 hours or 30 minutes to 1 hour. Methods for intravenous infusion of transferrin are known to persons of ordinary skill in the art, such as physicians, and can be implemented by such persons according to the patient's individual needs.

In accordance with the methods disclosed, proper dosages of a MI-Tf can be determined without undue experimentation using standard dose-response protocols. Exemplary doses of transferrin for human administration in accordance with the disclosure herein are from 25-150 mg/kg, 50-125 mg/kg, 75-100 mg/kg, or 85-115 mg/kg. These doses of transferrin are well tolerated without serious adverse events in this relatively ill patient population.

The MI-Tf can be administered, for example, daily, weekly, monthly or annually. Exemplary dosing regimens (courses) include, but are not limited to, daily for 7-21 days, daily for 10-14 days, every other day for 7-21 days, every other day for 10-14 days, every other day for 14-21 days, every other day for 14 days, every day for 10 days. Courses can also comprise dosing regimens wherein certain doses are administered at one interval and additional doses are administered at a second interval. For example, and not intended to be a limiting example, transferrin is administered daily for three days and then every other day for 10 days. Additionally, a course can be repeated periodically, for example, monthly, every other month, every three months, every four months, every five months or every six months. Courses can be repeated indefinitely.

In additional embodiments, each course can use the same or different doses of transferrin.

### EXAMPLES

### Example 1

### Exogenous Human Transferrin is Functional in Mouse Circulation

Iron is transported between sites of acquisition, storage, and utilization by serum transferrin (also referred to herein as “Tf”). Tf is predominantly synthesized by the liver; its main role is to deliver iron to cells by receptor-mediated endocytosis. Tf circulates in three forms: diferric-Tf (dTf, bound to two iron molecules), monoferric-Tf (mTf, bound to one iron molecule), and apo-Tf (unbound to iron) depending on available iron. The iron molecule can be located on N- or C-terminal binding site (lobe). The affinity of the transferrin receptor, TfR1, for dTf is greater than for mTf. However, the consequences of this greater affinity wane as iron supply is diminished. Typically, iron is bound to 30% of all Tf binding sites in circulation; mTf is normally the predominant form of Tf in circulation and increases further relative to dTf when Tf saturation is lowered. Each molecule of mTf delivers less iron than dTf. These combined effects result in less iron entering erythroid precursors when Tf saturation is low.

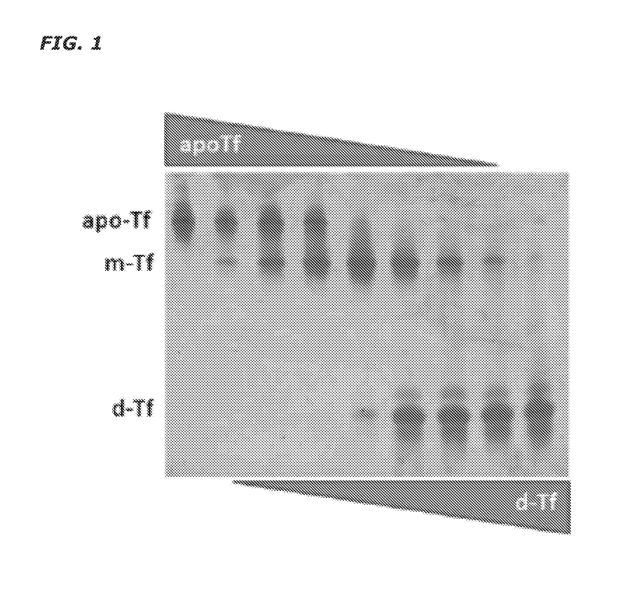

Incubating varying concentrations of apo-Tf and dTf in vitro generates mTf (FIG. 1). This observation demonstrates a dynamic transfer of iron from dTf to apo-Tf.

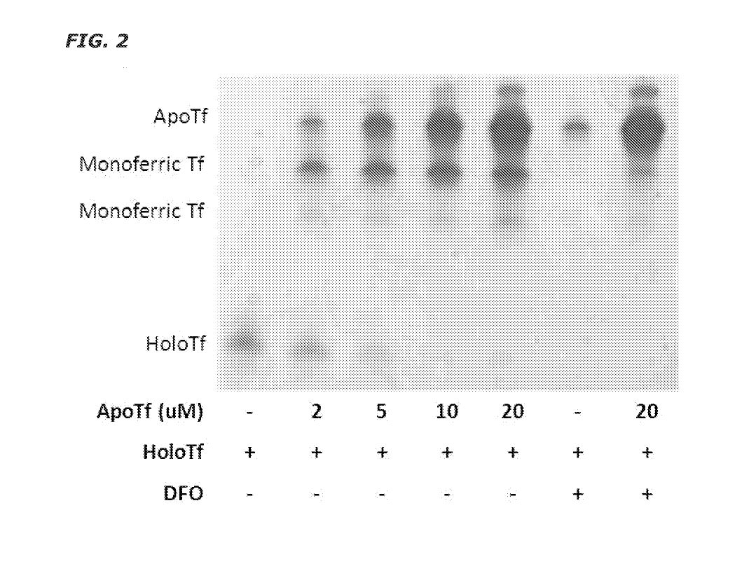

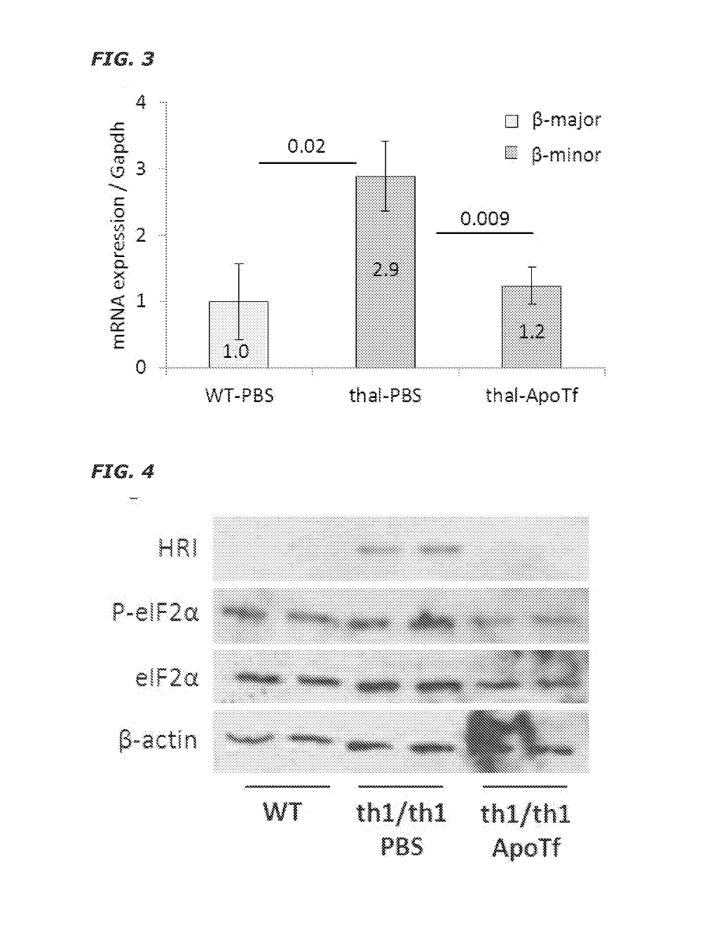

Injection of wild-type (WT) mice with a single intraperitoneal dose of apoTf (10 mg) decreases holoTf (P=0.01) and increases monoferric Tf (P=0.02) in the serum 6 hours after injection. Using both calcium mobilization and anti-Tf antibodies in flow cytometry, apoTf administration results in no TfR1 binding relative to holoTf in CHO cells (P<0.0001), and administration of a mixture of equal concentrations of apoTf:holoTf results in intermediate binding between apoTf and holoTf (P=0.004). A dose dependent decrease in cytosolic iron (P<0.001) and heme (P<0.01) is also observed in MEL cells treated with escalating concentrations of apoTf, an effect abrogated by the additional of DFO, an iron chelator. Urea gels of MEL cell supernatants reveal that monoferric Tf increases with increasing doses of apoTf and decreased in concurrently DFO treated cells (FIG. 2). These findings together suggest that changes in iron uptake result from increased monoferric Tf (rather than competition of apoTf for TfR1 binding sites). In addition, in vivo experiments demonstrated a decrease in heme concentration (56 vs. 67 μM, P<0.0001) in circulating RBCs as well as α-globin (1.3 vs. 3.3-fold, P=0.009) and β-globin (FIG. 3) mRNA expression in sorted bone marrow orthochromatophilic erythroblasts from apoTf- and PBS-treated th1/th1 mice. As heme is known to regulate globin expression through transcriptional and translational routes, bach1, heme oxygenase 1 (HO-1), and heme-regulated eIF2α kinase (HRI) were evaluated. No difference was seen in bach1 and HO-1 mRNA expression but a significant and unexpected decrease in HRI expression (1.0 vs. 2.8-fold, P=0.01) was seen in sorted bone marrow orthochromatophilic erythroblasts from apoTf− vs. PBS-treated th1/th1 mice (similar findings in all stages of terminal erythroid differentiation), suggesting that HRI is regulated by mechanisms independent of cellular iron and heme. Furthermore, Western blots of HRI, and its target eIF2α (total and phosphorylated), were also normalized in sorted bone marrow erythroid precursors from apoTf- and PBS-treated th1/th1 mice (FIG. 4). Taken together, this data suggests that exogenous apoTf in th1/th1 mice results in decreased cytosolic iron and heme as well as α- and β-globin synthesis as a consequence of increased monoferric Tf in circulation.

### Example 2

### Administration of Exogenous MI-Tf to Mice

Human, rather than mouse, transferrin is used for injection because it enables analysis of the quantities of each type of transferrin separately. Daily injections are employed in light of the 34-40 hr half-life of endogenous transferrin in mice and on the basis of prior experiments in hypotransferrinemic mice. The injected MI-Tf is in either the apotransferrin, monotransferrin, or holotransferrin form (if holotransferrin is used, at least one lobe should be locked so that the transferrin cannot release more than one iron molecule). The optimum dose of MI-Tf is determined by dose escalation experiments. Because maturation of committed precursors from erythroid colony-forming unit (CFU-E) stage to normoblast stage typically takes 7-10 days, initially mice are treated with transferrin for 10 days and analyzed. Mice are then treated with a 20-60 day course to represent a more chronic state of increased transferrin in the circulation.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A method for treating a disease or disorder associated with insufficient hepcidin expression or ineffective erythropoiesis in a subject in need thereof, comprising administering to said subject a therapeutically effective amount of a modified iron binding/releasing transferrin (MI-Tf), wherein the MI-Tf is a transferrin (a) capable of binding only one iron molecule or (b) capable of binding two iron molecules and releasing only one iron molecule.

2. (canceled)

3. (canceled)

4. The method of claim 1, wherein said MI-Tf comprises a first lobe and a second lobe, and wherein said first lobe binds iron, and wherein said second lobe has a decreased binding affinity for iron, wherein said lobe with a decreased binding affinity for iron comprises at least one amino acid insertion, deletion, or substitution, wherein said amino acid insertion, deletion, or substitution causes said MI-Tf to have decreased affinity for iron in said lobe.

5. (canceled)

6. The method of claim 4, wherein said lobe with a decreased binding affinity for iron does not bind iron.

7. (canceled)

8. The method of claim 1, wherein said MI-Tf comprises a first lobe and a second lobe, wherein both lobes bind iron, and wherein said first lobe has a decreased ability to release iron, wherein said lobe with a decreased ability to release iron comprises at least one amino acid insertion, deletion, or substitution, wherein said amino acid insertion, deletion, or substitution causes said MI-Tf to have decreased ability to release iron from said lobe.

9. (canceled)

10. The method of claim 1, wherein said MI-Tf comprises a first lobe and a second lobe, wherein said first lobe binds iron and has a decreased ability to release iron, and wherein said second lobe has a decreased binding affinity for iron.

11. The method of claim 1, wherein the method results in at least one effect in said subject selected from increased hepcidin expression, decreased non-transferrin bound iron (NTBI), reduced spleen size, decreased iron uptake by erythroid cells, increased erythroferrone, and decreased medullary or extramedullary erythropoiesis.

12.-15. (canceled)

16. The method of claim 1, wherein said MI-Tf comprises at least one amino acid substitution at a position selected from Y95, Y188, Y426, Y517, D63, G65, R124, Y45, T120, G394, E357, K511, D356, and H249 of the mature human transferrin amino acid sequence (SEQ ID NO:3).

17. The method of claim 16, wherein said at least one substitution is Y95F, Y188F, Y426F, Y517F, D63S, D63C, G65R, R124E, R124S, R124A, R124K, Y45E, T120A, G394R, E357A, K511A, D356A, or H249Q.

18. The method of claim 1, wherein said MI-Tf comprises amino acid substitutions at positions Y95 and Y188 of the mature human transferrin amino acid sequence (SEQ ID NO:3).

19. The method of claim 18, wherein said substitutions comprise Y95F and Y188F.

20. The method of claim 1, wherein said MI-Tf comprises amino acid substitutions at positions Y426 and Y517 of the mature human transferrin amino acid sequence (SEQ ID NO:3).

21. The method of claim 20, wherein said substitutions comprise Y426F and Y517F.

22.-39. (canceled)

40. The method of claim 1, wherein said MI-Tf is administered in a pharmaceutical composition comprising said MI-Tf and a pharmaceutically acceptable carrier.

41. A pharmaceutical composition comprising a therapeutically effective amount of:
an MI-Tf, wherein the MI-Tf is a transferrin (a) capable of binding only one iron molecule or (b) capable of binding two iron molecules and releasing only one iron molecule; and
a pharmaceutically acceptable carrier.

42. The pharmaceutical composition of claim 41, wherein said MI-Tf comprises a first lobe and a second lobe, and wherein said first lobe binds iron, and wherein said second lobe has a decreased binding affinity for iron, wherein said lobe with a decreased binding affinity for iron comprises at least one amino acid insertion, deletion, or substitution, wherein said amino acid insertion, deletion, or substitution causes said MI-Tf to have decreased affinity for iron in said lobe.

43. The pharmaceutical composition of claim 41, wherein said MI-Tf comprises a first lobe and a second lobe, wherein both lobes bind iron, and wherein said first lobe has a decreased ability to release iron, wherein said lobe with a decreased ability to release iron comprises at least one amino acid insertion, deletion, or substitution, wherein said amino acid insertion, deletion, or substitution causes said MI-Tf to have decreased ability to release iron from said lobe.

44. The pharmaceutical composition of claim 41, wherein said MI-Tf comprises a first lobe and a second lobe, wherein said first lobe binds iron and has a decreased ability to release iron, and wherein said second lobe has a decreased binding affinity for iron, wherein said lobe with a decreased binding affinity for iron comprises at least one amino acid insertion, deletion, or substitution, wherein said amino acid insertion, deletion, or substitution causes said MI-Tf to have decreased affinity for iron in said lobe, or to have decreased ability to release iron from said lobe.

45.-51. (canceled)

52. The pharmaceutical composition of claim 41, or wherein MI-Tf comprises at least one amino acid substitution at a position selected from Y95, Y188, Y426, Y517, D63, G65, R124, Y45, T120, G394, E357, K511, D356, and H249 of the mature human transferrin amino acid sequence (SEQ ID NO:3).

53. The pharmaceutical composition of claim 52, wherein said at least one substitution is Y95F, Y188F, Y426F, Y517F, D63S, D63C, G65R, R124E, R124S, R124A, R124K, Y45E, T120A, G394R, E357A, K511A, D356A, or H249Q.

54. (canceled)

55. The pharmaceutical composition of claim 54, wherein said substitutions comprise Y95F and Y188F.

56. (canceled)

57. The pharmaceutical composition of claim 56, wherein said substitutions comprise Y426F and Y517F.

58. (canceled)


SEQUENCE LISTING (3 sequences)

SEQ ID NO: 1 - DNA, 2808 bp, Homo sapiens
tggcaccgagcgagccgcgatgacaatggctgcattgtgcttcatgtcccttcccatcaacatttctgtgctggactccttccactcgcgggtcgtctccagagctcagaaaatgaggtgatcagtgggacgagtaaggaaggggggttgggagaggggcgattgggcaacccggctgcacaaacacgggaggtcaaagattgcgcccagcccgcccaggccgggaatggaataaagggacgcggggcgccggaggctgcacagaagcgagtccgactgtgctcgctgctcagcgccgcacccggaagatgaggctcgccgtgggagccctgctggtctgcgccgtcctggggctgtgtctggctgtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacctggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcgggagccttcaagtgtctgaaggatggtgctggggatgtggcctttgtcaagcactcgactatatttgagaacttggcaaacaaggctgacagggaccagtatgagctgctttgcctggacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtcccccccaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagaccttaaaatctcagaggtagggctgccaccaaggtgaagatgggaacgcagatgatccatgagtttgccctggtttcactggcccaagtggtttgtgctaaccacgtctgtcttcacagctctgtgttgccatgtgtgctgaacaaaaaataaaaattattattgattttatatttcaaaaactccattctttcctaaatattttcaacaaaggatttctttatgcattctgcctaaatacctatgcaactgagcccttccttctcagctcaagattcgtctggtctttccctacagctttgtgtgtgccatggccacatctcctgggtacagttcaaggagacatcttttctaaaagggtctgcgtgatcattaaaatataatcaaatgtaaaaaaaaaaaaaaaaaa

SEQ ID NO: 2 - protein, 698 aa, Homo sapiens
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 3 - protein, 679 aa, Homo sapiens
VPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP